(19) United States
(12) Patent Application Publication
(10) Pub. No.: US 2004/0137060 A1
(43) Pub. Date: Jul. 15, 2004

(54) Enalapril microspheres, dosage forms and chronotherapeutic methods based thereon

(76) Inventors: Siobhan Fogarty, Dublin (IE); Paul Maes, Potomac, MD (US)

Correspondence Address: CROWELL & MORING LLP, INTELLECTUAL PROPERTY GROUP, P.O. BOX 14300, WASHINGTON, DC, 20044-4300, US

(21) Appl. No.: 10/436,175

(22) Filed: May 13, 2003

Related U.S. Application Data
(60) Provisional application No. 60/379,395, filed on May 13, 2002.

(51) Int. Cl. A61K 9/26

(52) U.S. Cl. 424/469

(57) ABSTRACT

The invention provides coated particles containing: 1) monodispersed solid microspheres of Enalapril having a smooth spherical surface, and having a mean particle size of not greater than 500 microns prepared by a) subjecting a feedstock containing at least at least Enalapril as active ingredient and capable of being transformed to liquiform in the substantial absence of dissolving medium, to liquiflash conditions, which transform said feedstock from a solid to a liquid to a solid in less than five (5) seconds, said liquid characterized as having substantially unimpeded internal flow, and b) imparting shear force against flowing feedstock resulting from step “a)” in an amount sufficient to separate discrete particles by natural mass separation of said flowing feedstock in the presence of shear force impinging thereon while in said unimpeded-flow condition, wherein any active ingredient is substantially evenly distributed throughout the particles; and 2) at least one coating of a substance selected from the group consisting of controlled release and taste masking substances and wherein at least about 80% of the particles have a largest diameter of 60% of the mean diameter and the mean particle size is not greater than 500 microns.

### RELATED APPLICATIONS

This Application claims priority to Provisional Application No. 60/379,395, filed May 13, 2002, the contents of which are incorporated herein in their entirety.

### FIELD OF THE INVENTION

The present invention relates to microspheres containing Enalapril, dosage forms formed from the particles and chronotherapeutic methods using the microparticles and dosage forms.

### BACKGROUND OF THE INVENTION

Enalapril is an ACE inhibitor useful in treating hypertension and heart failure. It is currently marketed in the United States under the trademark VASOTEC (Enalapril maleate). It is disclosed and claimed in U.S. Pat. No. 4,374,829.

U.S. Pat. Nos. 4,374,829, 4,472,380 and 4,510,083 disclose methods useful in the preparation of Enalapril. The large-scale synthesis of Enalapril involves a key diastereoselective reductive amination reaction between an α-ketoester and alanylproline dipeptide. The reaction is catalyzed by Raney-Nickel (Ra-Ni). [Blacklock, T. J.; Shuman, R. F.; Butcher, J. W.; Shearin, W. E. Jr.; Budavari, J.; Grenda, V. J.; J. Org. Chem. 1988, 53, 836-844.]

The production of microspheres containing active agent(s) is described in U.S. Pat. No. 5,683,720, incorporated herein by reference. The patent discloses the use of liquiflash processing to spheronize compositions containing one or more active agents. As well, liquiflash particles are described in U.S. Pat. No. 5,849,223, which is also incorporated herein by reference.

The excessive stickiness caused by the use of binders, e.g., glycerine, is overcome by employing self-binding compositions. Self-binding, readily flowable compositions containing no glycerine are described in U.S. Pat. No. 5,840,331 and U.S. Ser. No. 08/914,972, filed Aug. 20, 1997.

One way to provide self-binding flowable formulations is to use shearform matrices or flosses. U.S. Pat. No. 5,587,172, incorporated herein by reference, discusses the use of flash heat techniques to produce sucrose-containing shearform flosses, which are then processed to yield quick-dissolving tablets.

The use of shearform matrices in forming comestible units is described in WO95/34290 (published Dec. 21, 1995) which is based on PCT application No. PCT/US95/07144, filed Jun. 6, 1995. WO95/34290 discloses a quick dissolving tablet which is formed by: (1) using flash-flow technology to provide a shearform matrix; (2) combining the partially recrystallized shearform matrix with an additive to form flowable, compactible particulate blends; and (3) compacting the blends at relatively low pressures to produce dosage forms, such as tablets.

Additionally, PCT publication WO 95/34293 (published Dec. 21, 1995) based on PCT Application No. PCT/US95/07194, filed Jun. 6, 1995, discloses a process and apparatus for making rapidly dissolving dosage forms by flash-flow processing. In WO 95/34293, a shearform matrix is formed by the flash-flow process, the shearform matrix is combined with an additive, and the matrix is molded to make a unit dosage form.

### SUMMARY OF THE INVENTION

The invention provides a new dosage form of Enalapril in a novel formulation utilizing Ceform™ Microsphere technology. The present invention provides enhanced dosage forms of Enalapril resulting in improved bioavailability, a better side effect profile and a novel dosing regime.

The invention provides compositions and shaped oral dosage forms made therefrom having improved properties. Among those properties are improved processability before shaping and enhanced dissolution and taste-masking properties when the dosage forms are used. In part, the advantages of the compositions of the invention relate to the size of the microspheres.

Various ingredients, such as coated microspheres containing active agent(s), are added, in suitable amounts, to the compositions of the present invention after the matrices are collected and chopped, but before they are shaped, e.g., by tabletting.

The present invention includes a method of making discrete particles by subjecting a solid organic feedstock, capable of being transformed to a liquiform in the substantial absence of a dissolving medium, to liquiflash conditions to provide substantially unimpeded internal flow. The feedstock, thus reduced to unimpeded internal flow, is subsequently discretized by natural mass separation of the flowing feedstock in the presence of shear force to form shearlite particles, i.e., particles which appear as shearform pearls.

During this rapid transition, shear forces can act on the material to discretize the feedstock. Thus, liquiflash conditions are the combination of temperature and force which induce an organic feedstock to flow and re-solidify into a changed shape as it is being discretized by the action of shear force. In preferred embodiments the size and the new shape are highly consistent among the discretized particles. Thus, the shape is preferably spheroidal and the size distribution is very limited with only minor variations.

In a preferred embodiment of the present invention, the feedstock material includes Enalapril so that the resulting product can be a delivery system for inclusion in a dosage unit.

The present invention also includes a method of making coated discrete particles comprising Enalapril which includes providing at least one coating to a discrete particle prepared by liquiflash processing. The discrete particles are prepared by subjecting a solid, organic feedstock to liquiflash conditions and separating discretize particles therefrom. The present invention also provides for the product resulting from coating such particles.

The medicaments contemplated for use in this aspect of the invention include Enalapril, alone or in combination with one or more active agents as set forth hereinbelow. Controlled-release substances are those which are known in the art, some of which have been set forth with specificity in the present disclosure. Taste-altering substances include taste-masking substances, sweeteners, flavorings, and, in general, any substance which changes the natural organoleptic perception of the product resulting from the liquiflash processing.

According to one aspect of the invention, there is provided a controlled-release Galenical preparation (such as a tablet and a capsule) of pharmaceutically acceptable Enalapril including the pharmaceutically acceptable salts thereof, such as the hydrochloride salt, suitable for evening dosing every 24 hours containing from about 1 mg to about 100 mg of the form of Enalapril associated with excipients to provide controlled (sustained) release of the form of Enalapril for providing a Cmax of Enalapril in the blood at between about 10 hours and about 15 hours (preferably about 11-about 13 hours) after administration, the preparation comprising the form of Enalapril in oral sustained-release dosage form in which the Enalapril is adapted to be released after administration over a prolonged period of time and the preparation is adapted to release the Enalapril.

### DETAILED DESCRIPTION OF THE INVENTION

Hypertension remains uncontrolled worldwide despite the availability of several classes of antihypertensive agents, including diuretics, α-blockers, β-blockers, calcium channel blockers, angiotensin-converting enzyme (ACE) inhibitors, angiotensin receptor blockers (ARB's) and a variety of central-acting agents. There is no currently available monotherapy that consistently reduces blood pressure over a full range of population subgroups with hypertension. The subject invention provides Enalapril based formulations that enhance blood pressure reduction alone or in combination with one or more active agents. Additionally, fixed dosage presentations of existing cardiovascular medications are an emerging option for hypertension therapy, providing patients already maintained on existing medications with convenient dosing options that further improve their compliance. The formulations of the invention help achieve the fixed dosage objective.

“Liquiflash conditions” as used herein means those conditions which provide transformation of a solid to a liquid state and then to the solid state (e.g., solid-liquid-solid) instantaneously. By instantaneously is meant less than seconds, in most cases on the order of fractions of a second, most preferably milli-seconds. Thus, certainly the transformation from solid to liquid to solid takes place in a time period of less than five seconds, preferably less than one second, and most preferably less than 0.1 seconds.

As a result of the process of the present invention, the discrete particles produced are preferably microspheres, which as used herein, preferably means not greater than about 500 μm, more preferably not greater than about 400 μm, and most preferably not greater than about 300 μm. In the preferred method of the present invention the liquiflash conditions are provided by a spinning head having a heated peripheral barrier with exit openings provided therethrough for passage of feedstock flowing under centrifugal force. The shear force referred to above is imparted to flowing feedstock by resistance of ambient atmosphere against the liquiform feedstock as it exits the spinning head.

The ambient atmosphere can be undisturbed except by the motion of the spinning head. Alternatively, the ambient atmosphere about the spinning head can be a positive counter or concurrent flow adjacent the outside surface of the processing barrier. This permits greater control of discretization of liquiform feedstock.

The discretized particles separated from the mass of flowing feedstock are cooled. In a preferred form of the present invention the discretized particles are monodispersed. “Monodispersed” as used herein refers to the production of a plurality of uniform spherical particulates, e.g., shearlites. As explained hereinabove methods for barrier processing of feedstock known in the art generally results in a product having a wide variety of sizes and shapes. This is due to many factors all of which contribute to a basic lack of control over the formation of particulates.

In the present invention, however, natural mass forming forces available in minute material masses, e.g., entropy, et al., provide a predictable uniform size. Thus, monodispersed means that at least about 40% by weight, preferably at least about 60% by weight and most preferably at least about 80% of the product herein have a largest diameter which is within 60% of the mean particle diameter. Particle diameter is the dimension which is the greatest straight line dimension in the largest plane taken through a three dimensional particulate. Generally, when the particulate is spheroidal in shape, the particulate diameter is the diameter of the spheroid. In a preferred embodiment, monodispersability means that at least 40% of the particulates are within 50% of the mean particulate diameter, and, in a most preferred embodiment, within 40% of the mean particulate diameter.

Processable feedstock materials used in the present invention are predominantly “organic material.” Organic material as used herein means carbon containing compounds, e.g., composition and structure of carbon containing compounds, whereas inorganic materials (or compounds) pertain to substances which do not contain organic type carbon. Polycarbon carbon compounds are preferably used in the present invention. Hydrocarbons are a major portion of organic materials, and are also preferred for use herein. Metals, inorganic carbonates and silicates, e.g., glass, are not considered organic materials for purposes of the present invention. Furthermore, proteinaceous material having high molecular weight is not considered “organic material” as used herein.

Unless otherwise stated, the parts and percentages used in the specification are weight percentages, based upon total composition weight. All disclosures referred to herein are hereby incorporated by reference.

The spheroidal liquiflash particles used in the invention generally contain at least Enalapril as the active agent, at least one solubilizer and at least one spheronization aid. Solubilizers are surfactants and other materials included in the microspheres to assist in the dissolution of the drug from the liquiflash particles. Polymeric surfactant materials containing one or more polyoxyalkylene blocks, such as poloxamers are typical. Useful solublilizers include Poloxamer 188 (also called “Pluronic F68”, from BASF Corp.) and other polyoxyethylene/polyoxypropylene copolymers. Pluronic F68 is preferred. Mixtures can be used. Spheronization aids are materials which help the drug-containing mix to form robust, durable spherical particles under liquiflash conditions. These spheronization aids include waxes and wax-like materials. Carnauba wax is preferred. When used, the carnauba wax acts as a binder, thus reducing the friability of the microspheres during handling and cooling. Mixtures are operable.

It is preferred that the liquiflash microspheres contain only active agent(s), solubilizer(s) and spheronization aid(s) and that they be coated after spheronization. It is also preferred that the coating used on the microspheres be one or more cellulosic coatings which serve to mask the taste of any unpleasant tasting active, or bio-affecting agent(s) in the microspheres. Useful cellulosic coatings include one or more alkylcellulose and/or hydroxyalkylcellulose polymers. Ethyl cellulose(EC)/-hydroxypropylcellulose (HPC) blends are useful, with EC:HPC ratios of 0.1:1 to 2:1 being useful. One preferred combination is a 1:1 blend of these polymers.

The cellulosic coatings are generally applied to the liquiflash particles after spheronization from organic solvent solution. Typical solvents include one or more of acetone, alkyl alcohols (e.g., isopropyl alcohol), and the like. Fluidized bed coating machines, e.g., the Glatt GPCG-60, and the like can be used. The coatings applied to the liquiflash particles may contain ingredients other than the cellulosics. Thus, one or more colorants, flavorants, sweeteners, controlled release agents and the like can also be used in the coating formulations. Other ingredients which may be mixed with the floss particles and coated liquiflash particles are fillers, fragrances, dyes, flavors, glidants, flow control agents, thickeners, sweeteners (both artificial and natural), and other conventional tablet additives. These ingredients are generally used in amounts of about 0.001% to about 20%, based upon total composition weight.

For example, fillers may be used to increase the bulk of the tablet. Some of the commonly used fillers are silicon dioxide (silica), calcium sulfate, both di- and tri-basic; starch; calcium carbonate; microcrystalline cellulose; modified starches, lactose, sucrose; mannitol and sorbitol. Flavors may be chosen from natural and synthetic flavoring liquids. An illustrative list of such agents includes volatile oils, synthetic flavor oils, flavoring aromatics, oils, liquids, oleoresins and extracts derived from plants, leaves, flowers, fruits, stems and combinations thereof. A non-limiting representative list of these includes citric oils, such a lemon, orange, grape, lime and grapefruit, and fruit essences, including apple, pear, peach, grape, strawberry, raspberry, cherry, plum, pineapple, apricot, or other fruit flavors. Grape flavoring is among the preferred flavors.

Other useful flavorings include aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodedenal (citrus, mandarin); mixtures thereof and the like.

The sweeteners may be chosen from the following non-limiting list: glucose (corn syrup), dextrose, invert sugar, fructose, and mixtures thereof (when not used as a carrier); saccharin and its various salts, such as the sodium salt; dipeptide sweeteners such as aspartame; dihydro-chalcone compounds, glycyrrhizin; Stevia Rebaudiana (Stevioside); chloro derivatives or sucrose such as sucralose; and sugar alcohols such as sorbitol, mannitol, xylitol, and the like. Also contemplated are hydrogenated starch hydrolysates and the synthetic sweeteners such as 3,6-dihydro-6-methyl-1-1-1,2,3-oxathiazin-4-one-2,2-dioxide, particularly the potassium salt (acesulfame-K), and sodium and calcium salts thereof. Acesulfame potassium is a preferred sweetener. Other sweeteners may be used, alone or in mixtures.

Other ingredients include binders, other than glycerine, which contribute to the ease of formation and general quality of the tablet. Binders include starches, pregelatinized starches, gelatin, polyvinylpyrrolidone, methylcellose, sodium carboxymethylcellulose, ethylcellulose, polyacrylamides, polyvinyloxoazolidone and polyvinylalcohols.

Color additives can be used in preparing tablets. Such color additives include food, drug and cosmetic colors (FD&C), drug and cosmetic colors (D&C) or external drug and cosmetic colors (Ext. D&C). These colors are dyes, lakes, and certain natural and derived colorants. Useful lakes include dyes absorbed on aluminum hydroxide or other suitable carriers.

Using the invention, strong, highly attractive dosage forms, e.g., tablets or lozenges, can be produced having textures and internal structures which are relatively open to solubilization. Applicants' compositions are preferably formed into tablets at pressures of from about 500 up to about 6,000 psi. using conventional tabletting machines, such as a rotary tablet press.

When chewable dosage forms are made, they are typically compressed to hardnesses of about 3 to about 15 SCU's, preferably about 4 to about 6. Tablet thicknesses for chewables are about 0.2 to about 0.3 inches. The following non-limiting examples illustrate the invention.

### EXAMPLE I

Coated Enalapril Microspheres: Enalapril powder was used in the following formulations to produce microspheres:

| | |
|---|---|
| 25% | 35% |
| Wt. Gain | Wt. Gain |

| Formula | % w/w | Mg | % w/w | Mg |
|---|---|---|---|---|
| Enalapril Maleate | 26.00 | 40 | 24.07 | 40 |
| Compritol 888 ATO | 42.00 | 64.62 | 38.89 | 64.62 |
| Milled Gelucire 50/13 | 12.00 | 18.46 | 11.11 | 18.46 |
| NE30D | 15.07 | 23.18 | 19.52 | 32.44 |
| Talc 400 | 3.02 | 4.64 | 3.91 | 6.49 |
| PEG8000 | 1.10 | 1.69 | 1.42 | 2.36 |
| Titanium Dioxide | 0.66 | 1.02 | 0.86 | 1.43 |
| Simethicone | 0.16 | 0.25 | 0.21 | 0.35 |
| Size 1 Capsule | — | — | — | — |

The microspheres selected were coated in a fluid bed coater for taste-masking at a 30% coating level with a coating solution containing a 1:1 ethylcellulose/-hydroxypropylcellulose blend in acetone:isopropyl alcohol solvent.

### EXAMPLE II

Unifloss: A preblend of 78.25% sucrose, 11.0% sorbitol, 10.0% xylitol and 0.75% TWEEN (Polysorbate) 80 was prepared. The floss preblend was processed using the 5″ crown head disclosed in U.S. Pat. No. 5,854,344, at a temperature of 250.degree. C. and rotational speed of 60 Hz (3600 rpm). The floss collected was chopped in the Littleford FKM600 mixer with 2% lactose (2% w/w of the floss) for 2 minutes at 100 rpm with the choppers on. 200 proof ethanol (0.5% based on weight of the floss) was sprayed on the chopped floss and mixed. The floss was then dried at 45.degree. C. for 90 minutes with intermittent mixing. The dried floss was screened through a 20 mesh screen.

### EXAMPLE III

Flash Dose Tablets: The microspheres (Example I) and the floss (Example II) were used in the following composition:

| Component | Percentage |
|---|---|
| Enalapril | 47.97 |
| taste-masked | |
| microspheres | |
| Floss | 48.88 |
| Grape flavor | 0.70 |
| Citric acid | 1.50 |
| Acesulfame potassium | 0.20 |
| Silicon dioxide | 0.25 |
| Sodium stearyl fumarate | 0.50 |

The coated Enalapril microspheres were blended with the sieved floss for 5 minutes in the Littleford FKM600 mixer, followed by the addition of flavors, sweeteners, and citric acid for another 3 minutes. Thereafter silicon dioxide was added and the mix blended for another 2 minutes. The final addition, sodium stearyl fumarate, was followed by blending for an additional 2 minutes.

What is claimed is:

1. A medicament comprising monodispersed microspheres of an active agent comprising Enalapril, each of said microspheres having a solid spherical body having active agent substantially evenly distributed throughout, wherein at least about 80% of the particles have a largest diameter of 60% of the mean diameter and the mean particle size is not greater than 500 microns.

2. The medicament of claim 1 wherein the microspheres show substantially no discontinuity therethrough.

3. A discrete particle comprising: monodispersed solid microspheres of Enalapril having a smooth spherical surface having a mean particle size of not greater than 500 microns prepared by:
a) subjecting a feedstock containing Enalapril and capable of being transformed to liquiform in the substantial absence of dissolving medium, to liquiflash conditions, which transform said feedstock from a solid to a liquid to a solid in less than five (5) seconds, said liquid characterized as having substantially unimpeded internal flow, and
b) imparting shear force against flowing feedstock resulting from step “a)” in an amount sufficient to separate discrete particles by natural mass separation of said flowing feedstock in the presence of shear force impinging thereon while in said unimpeded-flow condition, wherein any active ingredient is substantially evenly distributed throughout the particles and wherein at least about 80% of the particles have a largest diameter of 60% of the mean diameter and the mean particle size is not greater than 500 microns.

4. The discrete particle of claim 3 wherein the microsphere shave substantially no discontinuity therethrough.

5. The particles of claim 4 wherein said liquiflash conditions are provided by spinning said feedstock in a spinning head having a peripheral barrier provided with heating capability which delivers sufficient energy to said feedstock to liquify it instantaneously and with exit openings for passage of flowing feedstock therethrough in the presence of centrifugal force provided by spinning said head.

6. The particles of claim 5 wherein said shear force is imparted to said flowing feedstock by the resistance of ambient atmosphere against said flowing feedstock as it exits said spinning head.

7. The particles of claim 4 wherein said discretized separated particles are cooled upon exiting said spinning head.

8. The particles of claim 4 wherein said feedstock comprises an additional medicament other than Enalapril.

9. Coated particles comprising:
1) monodispersed solid microspheres of Enalapril having a smooth spherical surface having a mean particle size of not greater than 500 microns prepared by:
a) subjecting a feedstock containing at least at least Enalapril as active ingredient and capable of being transformed to liquiform in the substantial absence of dissolving medium, to liquiflash conditions, which transform said feedstock from a solid to a liquid to a solid in less than five (5) seconds, said liquid characterized as having substantially unimpeded internal flow, and
b) imparting shear force against flowing feedstock resulting from step “a)” in an amount sufficient to separate discrete particles by natural mass separation of said flowing feedstock in the presence of shear force impinging thereon while in said unimpeded-flow condition, wherein any active ingredient is substantially evenly distributed throughout the particles; and
2) at least one coating of a substance selected from the group consisting of controlled release and taste masking substances and wherein at least about 80% of the particles have a largest diameter of 60% of the mean diameter and the mean particle size is not greater than 500 microns.

10. The particle of claim 9 wherein surface the microspheres have substantially no discontinuity therethrough.

11. The particle of claim 9 wherein said feedstock comprises a saccharide-based material.

12. The particle of claim 10 wherein said saccharide-based feedstock comprises a sugar.

13. An oral dosage form produced by shaping a composition comprising:
(a) coated liquiflash particles containing about 20.0 to about 90.0% Enalapril, about 7.0 to about 10.0% carnauba wax, and about 7.0 to about 10.0% Pluronic F68; and
(b) glycerine-free bodies containing about 75.0 to 85.0% sucrose, about 8.0 to about 15.0% sorbitol, and about 5.0 to about 15.0% xylitol.

14. An oral dosage form produced by shaping a composition comprising:
(a) coated liquiflash particles containing 85% Enalapril, 7.5% carnauba wax, and 7.5% Pluronic F68; and
(b) glycerine-free bodies containing 78.25% sucrose, 11.00% sorbitol, 10.00% xylitol and 0.75% Polysorbate 80.

15. The dosage form of claim 14 containing approximately: 48% coated liquiflash particles, 48.85% glycerine-free bodies, 0.70% flavor, 1.5% citric acid, 0.2% sweetener, 0.25% silicon dioxide, and 0.50% sodium stearyl fumarate.

16. A process for preparing fast dissolving dosage forms comprising the steps:
(1) providing liquiflash particles containing Enalapril as active agent, solubilizers and spheronization aid(s),
(2) coating the particles of step (1) with a cellulosic taste-making material,
(3) blending the coated particles of step (2) with glycerine-free bodies, and
(4) shaping the blend to produce dosage forms.
